(19) United States
(12) Patent Application Publication
(10) Pub. No.: US 2025/0288677 A1
(43) Pub. Date: Sep. 18, 2025

(54) MULTIPHASIC LOCALIZED IN VIVO MOLECULAR DELIVERY

(71) Applicant: UNIVERSITY OF SOUTH FLORIDA, Tampa, FL (US)

(72) Inventors: Anna Bulysheva, Tampa, FL (US); Mark Jeffrey Jaroszeski, Tampa, FL (US); Richard Heller, Tampa, FL (US)

(21) Appl. No.: 19/080,387

(22) Filed: Mar. 14, 2025

Related U.S. Application Data
(60) Provisional application No. 63/565,371, filed on Mar. 14, 2024.

(51) Int. Cl. A61K 41/00; A61K 39/00

(52) U.S. Cl. CPC A61K 41/0047; A61K 39/0011; A61K 2039/53

(57) ABSTRACT

The present disclosure relates to methods for transferring a nucleic acid in vivo into mammalian cells by biphasic electrotransfer. Injection via syringe or needle is accompanied by a biphasic pulse of high voltage, resulting in significantly reduced stimulation of cells.

### CROSS REFERENCE TO RELATED APPLICATIONS

This application claims the benefit of and priority to U.S. Provisional Application Ser. No. 63/565,371, filed Mar. 14, 2024, the entirety of which is incorporated herein by reference.

### BACKGROUND

Gene electrotransfer (GET), a form of reversible electroporation, is a method of gene delivery used to introduce genetic information to various mammalian tissues in vivo. This method uses pulsed electric fields acting on cell membranes to facilitate transfer of genetic material from the interstitial space outside the cells, into the cells and inside cell nuclei. The use of a pulsed electric field allows the introduction of genetic material into the cells, without the use of viral vectors. GET has become popular as a delivery method for a variety of applications such as cancer treatment, nucleic acid based vaccines, tissue regeneration and tissue repair.

### SUMMARY

Disclosed herein is a method to address the issue of muscle contractions in a patient that occur as a result of monophasic GET pulse sequences.

In one embodiment, the method consists of delivery of asymmetric, biphasic pulse sequences that enhance gene delivery to tissues, while reducing stimulation of skeletal muscle thus reducing muscle contraction.

In another embodiment, the method can be administered using a variety of electrode configurations.

In another embodiment, the method utilizes a monopolar configuration of electrode. Other embodiments of the method include use of a “basic bipolar” electrode (two electrodes: 1 negative and 1 positive). Another embodiment of the method utilizes 4 or more electrodes in various geometric configurations including but not limited to square, circle, triangle, and rectangle.

In another embodiment, the pulsing sequence includes at least one train of biphasic asymmetric pulses, where each train includes a positive and a negative phase with one phase being a long pulse width and one phase at a higher amplitude (voltage).

In another embodiment, a specific pulsing sequence is determined for each therapeutic application and each tissue type. Various implementations of the disclosure further provide a method for generating an electroporation waveform.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows a representative asymmetric biphasic pulse sequence.

FIG. 2 shows that asymmetric, biphasic pulses are non-stimulating in a guinea pig model of gene delivery to the skin. Stars indicate muscle twitching due to applied pulses.

FIGS. 3A-3C show representative asymmetric biphasic pulse sequences where muscle stimulation is reduced due to asymmetric biphasic pulse shape and not due to speed of the delivered pulses. (A) Muscle stimulation in asymmetric biphasic pulses showing reduced muscle stimulation/twitching compared to FIGS. 3B and 3C. (B) Traditional monophasic pulses showing increased muscle stimulation/twitching compared to FIG. 3A. (C) Monophasic pulses at the same magnitude, frequency and duration as the asymmetric pulses in FIG. 3A, showing increased muscle stimulation/twitching. A monopolar electrode was applied to each treatment site on the skin of Sprague Dawley rats, with independent accelerometers measuring movement of fore-and hind limbs which were allowed to move freely, n=8.

FIG. 4 shows the positive (+), negative (−), and off states of a circuit in the biphasic pulse delivery system.

### DEFINITIONS

As used herein, the term “train” refers a sequence of GET pulses.

As used herein, the term “non-stimulating” means reduced muscle twitching compared to twitching observed due to a monophasic pulse.

### DETAILED DESCRIPTION

The foregoing and other embodiments and advantages of the present disclosure will appear from the following description. In the description, reference is made to the accompanying drawings that form a part hereof, and in which there is shown by way of illustration one or more exemplary versions. These versions do not necessarily represent the full scope of the disclosure.

Gene delivery methods in vivo can be classified into viral and nonviral categories. Viral vectors are highly efficient in gene transfer; however, serious concerns regarding the possibility of insertional mutagenesis and induction of the host immune response limit the desirability of the use of viral vectors as a means of gene delivery. Among nonviral techniques, intramuscular injection of naked DNA has been demonstrated to be a safe, simple, and inexpensive approach for gene delivery. However, this method also has several major limitations. First, the level of gene expression is in most cases too low for efficient treatment of diseases that require a high level of expression. Therefore, this method shows promise only for low-expression applications such as vaccination. Second, there is high interindividual variability in the level of the transferred gene expression. In addition, the age and species of animal appear to influence the effectiveness of the technique, with younger animals showing higher transfection activity than older ones.

Gene electrotransfer (GET) has emerged as a powerful method of DNA delivery offering several medical applications, including DNA vaccination and gene therapy. GET entails the application of electric fields to cells which then experience a local and transient change of membrane permeability. While early GET protocols showed low efficiency of gene expression in vivo, progress has been made to improve the efficiency of GET, including establishment of pulsing protocols, as well as pulser and electrode design. The advancement has concentrated on monophasic pulse sequences on the order of micro and milli-second long pulses. Other advancements include, improved electrode design, such as multielectrode arrays, four plate electrodes, monopolar electrodes, moderate heat application, and impedance monitoring. However, all reported monophasic GET pulse sequences result in muscle contractions and muscle stimulation. This stimulation can range from mild discomfort to an increased risk of ventricular fibrillation. Therefore, there exists a need for a non-stimulating system that allows for wider adoption of GET for a multitude of gene therapy applications, which would reduce both risk and discomfort to patients.

In one embodiment, provided herein is a method of in vivo delivery of a nucleic acid into cells of a patient in need thereof by gene electrotransfer.

In one embodiment, provided herein is a method of in vivo delivery of a nucleic acid into cells of a patient wherein the nucleic acid is injected by any means including by syringe or by needle.

In another embodiment, a method is provided wherein the gene electrotransfer consists of a biphasic electrical pulse.

In another embodiment, a method is provided wherein the biphasic pulse is non-stimulating to the patient.

In another embodiment, a method is provided wherein the biphasic pulse is minimally stimulating to the patient.

In another embodiment, a method is provided wherein the biphasic pulse reduces stimulation of cells in the patient by 2-fold, 3-fold, 4-fold and 5-fold, when compared to stimulation of cells with a symmetric or asymmetric monophasic pulse.

In another embodiment, a method is provided wherein the biphasic pulse of voltage has a strength selected from the group consisting of 1 to 150 V, 1 to 10 V, 10 to 20 V, 20 to 30 V, 30 to 40 V, 40 to 50 V 50 to 60 V, 60 to 70 V, 70 to 80 V, 80 to 100 V, 100 to 120 V and 120 to 150 V.

In another embodiment, a method is provided wherein the duration of the biphasic pulse is from 0.1 to 50 μs (microseconds).

In another embodiment, a method is provided wherein the duration of the biphasic pulse is selected from the group consisting of from 0.01 to 1 μs, from 1 to 3 μs, from 3 to 5 μs, from 4to 6 μs, from 5 to 7 μs, from 7 to 10 μs, from 10 to 15 μs, 15 to 20 μs, 20 to 30 μs, 30 to 40 μs and from 40 to 50 μs.

In another embodiment, a method is provided wherein the electrodes used to deliver the biphasic pulse are non-penetrating electrodes.

In another embodiment, a method is provided wherein the electrodes used to deliver the biphasic pulse are penetrating electrodes.

In another embodiment, a method is provided wherein the electrodes used to deliver the biphasic pulse are caliper electrodes.

In another embodiment, a method is provided wherein the electrodes used to deliver the biphasic pulse are monopolar electrodes.

another embodiment, a method is provided wherein the electrodes used to deliver the biphasic pulse are pin electrodes.

In another embodiment, a method is provided wherein the electrodes used to deliver the biphasic pulse are needle electrodes.

In another embodiment, a method is provided wherein the biphasic pulse is delivered to cells selected from the group consisting of mammalian cells including but not limited to skin cells, blood cells, muscle cells, fat cells, nerve cells, tumor cells, cardiac cells, connective tissue cells, and epithelial cells.

In another embodiment, a method is provided wherein the nucleic acid delivered in the biphasic pulse encodes an antigenic or immunogenic protein.

In another embodiment, a method is provided wherein the nucleic acid delivered in the biphasic pulse encodes an antigen including but not limited to tumor antigen, a viral antigen or a bacterial antigen.

In another embodiment, a method is provided wherein the nucleic acid delivered in the biphasic pulse encodes an antigenic protein for reducing, preventing or suppressing a tumor.

In another embodiment, a method is provided wherein a patient in need has a disease selected from the group consisting of diabetes, cancer, metabolic disorder, infectious disease, vascular disease, heart failure, atrial fibrillation, wound healing, protein replacement therapy, regenerative medicine applications and blood clotting disorder.

In another embodiment, a method is provided wherein the nucleic acid delivered in the biphasic pulse is a single-stranded or double-stranded RNA.

In another embodiment, a method is provided wherein the nucleic acid delivered in the biphasic pulse is a single-stranded or double-stranded DNA.

In another embodiment, a method is provided wherein the nucleic acid is provided for vaccine therapy or gene therapy to a patient in need.

In another embodiment, a method is provided wherein the nucleic acid provided for vaccine therapy or gene therapy to a patient in need, encodes an antigenic or immunogenic protein.

In another embodiment, a biphasic pulse delivery system is described that delivers square pulse sequences, which is a non-stimulating, handheld portable device that does not require extensive specialized training or the use of anesthesia, allowing a clinician to operate in an outpatient setting.

In another embodiment, the biphasic pulse delivery system uses an h-bridge topology.

In another embodiment, the biphasic pulse delivery system allows pulses to travel in either direction across the target tissue.

In another embodiment, the circuit of the biphasic pulse delivery system includes two source voltages, rather than the typical single source, to allow for asymmetric pulsing.

In another embodiment, the biphasic pulse delivery system uses reduced voltage and pulse width, providing a reduction in stimulation events for the subject, specifically in irreversible electroporation (IRE).

In another embodiment, the biphasic pulse delivery system utilizes a Gallium Nitride Field Effect Transistor (GaNFET).

As used herein, the term “subject” may be used interchangeably with the term “patient” or “individual” and may include an “animal” and in particular a “mammal.” Mammalian subjects may include humans and non-human animals, such as other primates, domestic animals, farm animals, and companion animals such as dogs, cats, guinea pigs, rabbits, rats, mice, horses, cattle, cows, etc.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “prevent,” “preventing,” “prevention,” “prophylactic treatment” and the like refer to reducing the probability of developing a disease, disorder or condition in a subject, who does not have, but is at risk of or susceptible to developing a disease, disorder or condition.

Treating cancer in a subject includes the reducing, repressing, delaying or preventing cancer growth, reduction of tumor volume, and/or preventing, repressing, delaying or reducing metastasis of the tumor. Treating cancer in a subject also includes the reduction of the number of tumor cells within the subject. The term “treatment” can be characterized by at least one of the following: (a) reducing, slowing or inhibiting growth of cancer and cancer cells, including slowing or inhibiting the growth of metastatic cancer cells; (b) preventing further growth of tumors; (c) reducing or preventing metastasis of cancer cells within a subject; and (d) reducing or ameliorating at least one symptom of cancer. In some embodiments, the optimum effective amount can be readily determined by one skilled in the art using routine experimentation. The aim of treatment includes the alleviation or prevention of symptoms, slowing or stopping the progression or worsening of a disease, disorder, or condition and/or the remission of the disease, disorder or condition. The term “effective amount” or “therapeutically effective amount” refers to an amount sufficient to effect beneficial or desirable biological and/or clinical results.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter. Embodiments recited as “including,” “comprising,” or “having” certain elements are also contemplated as “consisting essentially of” and “consisting of” those certain elements.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and embodiment of a described embodiment or feature contained in the same, or a definitive decision to use a specific skill regarding a described embodiment or feature contained in the same. In this latter context, the modal verb “may” has the same meaning and connotation as the auxiliary verb “can.”

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred embodiments of this invention are described herein, including the best mode known to the inventors for carrying out the invention. Variations of those preferred embodiments may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Methods

### Animals

Female Hartley guinea pigs weighing approximately 250-300 g were used for this study. All experimental studies followed an approved University of South Florida Institutional Animal Care and Use Committee protocol, in accordance with the Guide for the Care and Use of Laboratory Animals at an AAALAC-accredited facility. Animals were quarantined for and acclimated for a 7-day period before any procedures were conducted.

### Plasmid DNA

Plasmid DNA encoding luciferase, Nanoplasmid™M was purchased from Aldevron (Fargo, ND). Plasmid DNA was suspended in sterile saline at 2 mg/mL by Aldevron. Endotoxin levels were below 0.1 EU/ug plasmid, confirmed by Aldevron via a Limulus Amebocyte Lysate assay.

### Gene Electrotransfer

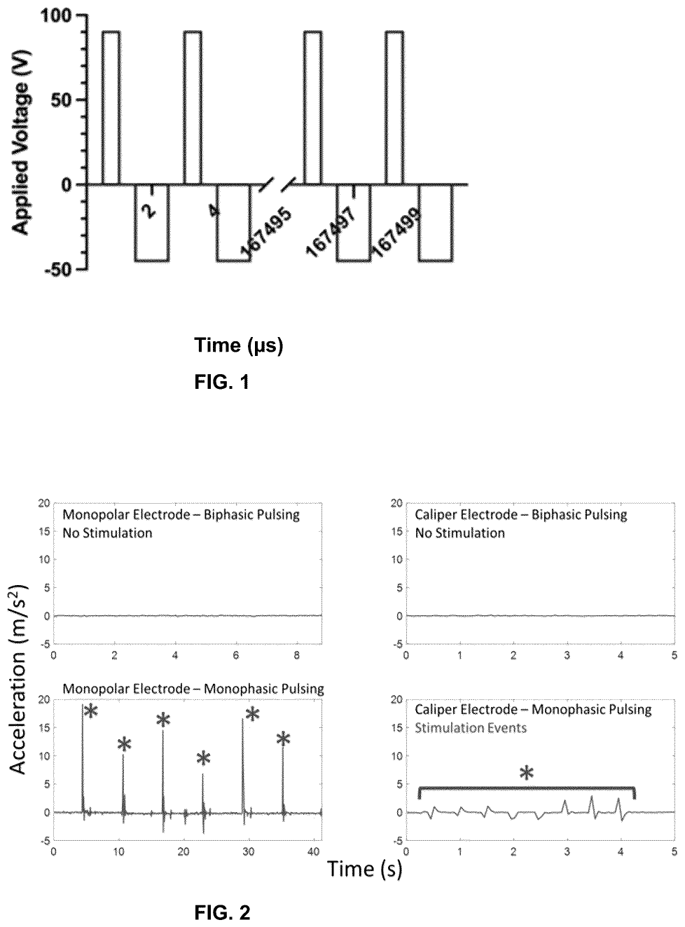

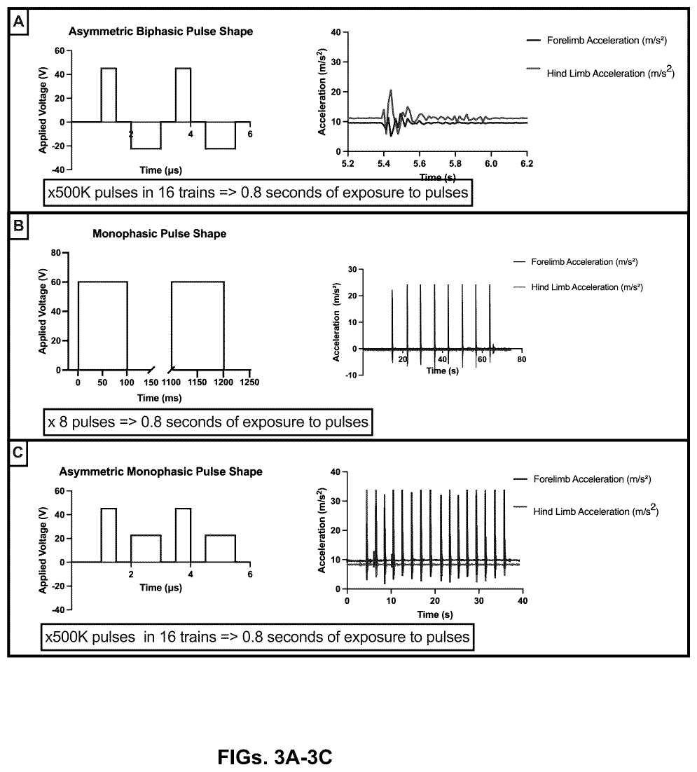

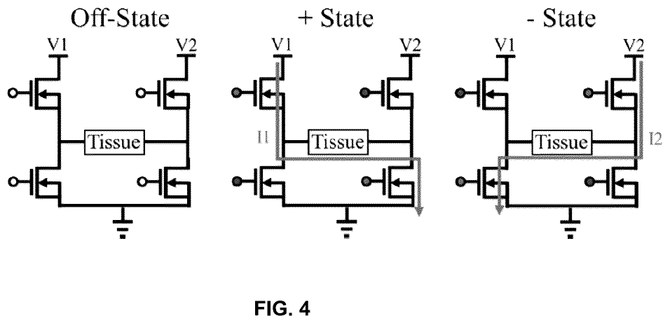

Each treatment site received a 50 μL injection of 2 mg/mL plasmid DNA. Sites were then immediately pulsed according to pulsing parameters in Table 1. An example asymmetric, biphasic pulse sequence is shown in FIG. 1, with a period under 2 μs, and the overall time that tissue was exposed to the biphasic pulse was equivalent to the pulse duration of an effective monophasic pulse to achieve delivery. For example, it would take ˜67,000 biphasic pulses in FIG. 1, to reach a pulse duration of 100 ms pulse typically used for monophasic delivery to muscle. Three different electrode designs were used. A caliper electrode or a four-plate non-penetrating electrode (4PE) were used, with a gap of 0.5 cm between paired electrodes. Additionally, a 10 mm monopolar electrode was also used to compare monophasic and biphasic pulse sequences. Pulses were applied using commercially available pulse generators adapted to apply designated pulsing sequences. The number of treatment sites per experimental group was n=4. There were up to 6 treatment sites on the flanks per animal depending on the size of the animal, to ensure a 1-2 cm gap between the treatment sites. Muscle twitching was monitored using a small, insulated accelerometer placed ˜1 cm away from the treatment site. The conditions for treatment sites were randomized, to control for variability between animals.

### Bioluminescence Imaging

Animals were imaged for bioluminescence on days 2, 7, 14, and 21. Each animal was anesthetized and received a subcutaneous injection of D-luciferin (Gold Biotechnology, Inc., St. Louis, MO). The in vivo Imaging System (PerkinElmer, Akron OH) was used to capture and quantitate bioluminescence signal. Groups were compared with an ordinary two-way ANOVA, and Tukey's multiple comparisons test, with p<0.05 considered significant.

### Reporter Gene Delivery

Luciferase reporter gene expression was evaluated in guinea pigs as shown in Table 1.On day 2 bioluminescence levels yielded comparable expression enhancement with biphasic and monophasic conditions over controls, showing positive results for enhancing gene delivery and expression.

TABLE 1
Asymmetric, biphasic pulses enhance luciferase gene expression in guinea pig skin on day 2.

| | | | | | Number of | | Standard |
|---|---|---|---|---|---|---|---|
| | | Applied | Pulse | Pulse | Pulse | Average Flux | error of |
| Waveform | Electrode | Voltage | Length | Number | Trains | (p/s) | the mean |
| Biphasic | Monopolar | −90 V/45 V | 1 μs/2 μs | 22300 | 24 | 1.60E+07 | 4.85E+06 |
| Biphasic | Monopolar | −90 V/45 V | 0.5 μs/1 μs | 22300 | 36 × 2 | 4.07E+07 | 1.99E+07 |
| Injection Only | N/A | N/A | N/A | N/A | N/A | 5.55E+07 | 4.19E+07 |
| Biphasic | Monopolar | −90 V/45 V | 1 μs/2 μs | 22300 | 24 × 2 | 8.40E+07 | 7.25E+07 |
| Monophasic | Monopolar | −90 V | 100 ms | 8 | 1 | 8.67E+07 | 2.39E+07 |
| Biphasic | 4PE | −90 V/45 V | 1 μs/2 μs | 22300 | 18 | 9.83E+07 | 4.14E+07 |
| Biphasic | 4PE | −90 V/45 V | 0.5 μs/1 μs | 22300 | 18 | 1.12E+08 | 3.39E+07 |
| Biphasic | 4PE | −90 V/45 V | 0.5 μs/1 μs | 22300 | 24 | 1.13E+08 | 7.14E+07 |
| Monophasic | 4PE | −90 V | 150 ms | 8 (4 × 4) | 1 | 1.20E+08 | 6.13E+07 |
| Biphasic | 4PE | −90 V/45 V | 1 μs/2 μs | 22300 | 24 | 1.21E+08 | 3.73E+07 |
| Biphasic | Monopolar | −90 V/45 V | 0.5 μs/1 μs | 22300 | 36 | 1.82E+08 | 1.53E+08 |

### Results-Skeletal Muscle Stimulation

Biphasic, asymmetric pulse sequences significantly reduced muscle twitching using both monopolar and caliper electrodes compared to corresponding monophasic pulse sequences with equivalent total pulse duration and applied voltage, as shown in FIG. 2. Eight pulse trains of 67,000 biphasic pulses were applied via monopolar and caliper electrodes with zero detected muscle twitching, as shown in the upper panels of FIG. 2, compared to significant muscle stimulation and twitching of up to 20 m/s² detected for the monophasic pulses, as shown in the lower panels of FIG. 2.

What is claimed is:

1. A method of in vivo delivery of a nucleic acid into cells of patient in need thereof, wherein the nucleic acid is injected by any means including syringe or needle, and is electrically transferred into a patient's cells by delivering an asymmetric, biphasic pulse consisting of 0.01 to 100 V/cm and a duration of 0.1 to 10 microseconds (μs) and;
wherein the biphasic pulse causes minimal stimulation to the patient.

2. The method according to claim 1, wherein the biphasic pulse of voltage has a strength of between 1 and 150 V.

3. The method according to claim 1, wherein the duration of the biphasic pulse is 0.01 to 50 μs.

4. The method according to claim 1, wherein the electrodes used are non-penetrating electrodes.

5. The method according to claim 1, wherein the electrodes used are penetrating electrodes.

6. The method according to claim 1, wherein the electrodes used are caliper electrodes.

7. The method according to claim 1, wherein the electrodes used are monopolar electrodes.

8. The method according to claim 1, wherein the electrodes used are needle electrodes.

9. The method according to claim 1, wherein the electrodes used are pin electrodes.

10. The method according to claim 1, wherein the cells are selected from the group of mammalian cells including but not limited to skin cells, blood cells, muscle cells, cardiomyocytes, fat cells, nerve cells, stem cells, connective tissue cells and epithelial cells.

11. The method according to claim 1, wherein the nucleic acid encodes an antigenic or immunogenic protein.

12. The method according to claim 1, wherein the patient has a disease selected from the group consisting of diabetes, cancer, metabolic disorder, infectious disease, vascular disorder, heart failure, atrial fibrillation, wound healing, protein replacement disorders, regenerative medicine applications and blood clotting disorder.

13. The method according to claim 1, wherein the biphasic pulse reduces stimulation of cells in the patient by 2-fold, 3-fold, 4-fold and 5-fold, when compared to stimulation of cells with a symmetric or asymmetric monophasic or pulse.

14. The method according to claim 11, wherein the nucleic acid encodes a tumor antigen, a viral antigen or a bacterial antigen.

15. The method according to claim 11, wherein the nucleic acid encodes an antigenic protein for reducing, preventing or suppressing a tumor.

16. The method according to claim 1, wherein the nucleic acid is a single-stranded or double-stranded RNA.

17. The method according to claim 1, wherein the nucleic acid is a single-stranded or double-stranded DNA.

18. A method of vaccination or gene therapy in a subject, wherein the method comprises administering a nucleic acid according to claim 1.

19. The method of vaccination according to claim 15, wherein the nucleic acid encodes an antigenic or immunogenic protein.
